(19) United States
(12) Patent Application Publication
(10) Pub. No.: US 2023/0302170 A1
(43) Pub. Date: Sep. 28, 2023

(54) SYSTEM AND METHOD FOR CLEANING AND DISINFECTING A DENTAL INSTRUMENT

(71) Applicant: Bien-Air Holding SA, Bienne (CH)

(72) Inventors: Davide SARCHI, Zürich (CH); Marco GALLINA, Gletterens (CH); Edgar SCHÖNBÄCHLER, Neuchâtel (CH)

(21) Appl. No.: 18/039,990

(22) PCT Filed: Dec. 3, 2021
(86) PCT No.: PCT/EP2021/084229

§ 371 Date: Jun. 2, 2023

(30) Foreign Application Priority Data
Dec. 3, 2020 (EP) 20211659.6

(51) Int. Cl. A61L 2/07; A61L 2/10

(52) U.S. Cl. CPC A61L 2/07; A61L 2/10; A61L 2202/14; A61L 2202/24

(57) ABSTRACT

The present invention relates to a system (1000) for cleaning and/or disinfecting a tool (600) for dental or surgical use, comprising a steam generator (100) and a compressed air source (200), the cleaning and/or disinfecting system (1000) further comprising a flow regulating device (400) designed to inject wet steam (1) and compressed air (2) into at least one channel of the tool (600) for dental or surgical use that is to be treated.

### TECHNICAL FIELD OF THE INVENTION

The present invention relates to the field of instruments and devices for practitioners in the medical field, such as dentists and dental surgeons. More specifically, it relates to a system and method of steam cleaning and disinfection for devices of the handpiece or dental turbine type.

### STATE OF THE ART

Dentists, hygienists and dental surgeons, as well as other practitioners in the field of endodontics, periodontics, oral surgery or implantology, often use various instruments and devices, the most common of which are straight handpieces, contra-angles (so called because of their slight curvature) and air turbines. These devices generally include a head, which accommodates a rotor and allows to transfer the movement to the milling, cutting or polishing tool and which goes into the patient's mouth, and a handle, i.e. the component that is manipulated by the dental surgeon. The straight handpieces and contra-angles are connected to an electric motor (micromotor) which provides the mechanical energy necessary for the movement of the rotor and thus of the milling, cutting or polishing tool. Air turbines are simply connected to a tube that provides the compressed air to set the rotor in motion. The system of coupling an air turbine to a tube is very similar to the coupling system allowing the coupling of a handpiece or a contra-angle to a motor.

The motor part is mounted on the handpiece via a standardized coupling nose for transmitting the motor's transmission torque or for routing a compressed air flow that sets the turbine in rotation, and at the same time the handpiece and the motor part usually further comprise a number of channels that are connected to one another, into which fluids such as compressed air and/or water are able to be conveyed for cooling the device or for spraying the area of operation during utilization. An example of a cooling and spray channel arrangement is mentioned, for example, in the patent document EP 1,768,597.

For the cleaning and disinfection of such instruments of dental handpiece type, the following operations are usually performed in automatic sequence or according to the user's settings:
first of all, pre-cleaning, cleaning or rinsing with cold, warm or hot water; followed by a
cleaning and/or disinfection with the aid of chemical substances; and finally
debris removal and/or drying by means of the application of compressed air.

The patent document U.S. Pat. No. 5,723,090 describes a solution employing such a method for the cleaning and the disinfection of handpieces.

Alternatively or in addition to the second step of the sequence described below, a cleaning and/or disinfection method using saturated or over-heated steam is furthermore known, for example from document US 2012298151. When these two conditions are fulfilled, we refer to dry steam.

A final lubrication step with oil and possibly air or a mixture of both can then be provided.

The American document U.S. Pat. No. 6,368,556 B1 describes another cleaning and sterilization solution for dental handpieces in an autoclave, according to which a fixing support replacing the motor part is provided with separate channels for cleaning respectively the spray with steam or hot air and respectively the rotating parts with oil and hot air; the sterilization process uses here again dry steam at 122° C. under pressure conditions of 1.1 bar in the autoclave.

A major disadvantage of these solutions known in the prior art is that the cleaning of the interior of the devices is not satisfactory, because it may result in an accumulation of solid and/or organic residues (e.g. blood and proteins) in the gears, ball bearings etc. In addition, the accumulation of residues makes any other disinfection and/or sterilization procedure, including steam sterilization in an autoclave, increasingly ineffective, and can also lead to accelerated aging of mechanical devices and the risk of overheating and burning of the patient.

The inefficiency of washing procedures using water is mainly related to the fact that water drops are unable to penetrate the gaps and hollow areas inside the devices; the addition of enzymatic or alkaline chemical substances may accelerate the dissolution of residues reachable by water drops, but does not improve the access to hollow areas.

The ineffectiveness of alternative cleaning procedures using saturated or superheated steam is related to the fact that heat causes protein denaturation, making it more difficult for dirt and biofilms to dissolve and detach from surfaces.

Moreover, disinfection via saturated or superheated steam injection (of dry-steam type) is not universally effective, because the high pressure and high temperature conditions of the steam at the inlet of the device are not maintained throughout the entire device. Indeed, the temperature gradient between the inlet (generally positioned at the back of the device, at the level of the nozzle replacing the motor part for cleaning and disinfection) and the outlet (positioned in the head of the device), causes the condensation of the steam on the inner surfaces of the device, especially at the level of the head; this strongly limits the bactericidal and fungicidal capacity in the most contaminated areas of the dental device, which is precisely at the level of the head of the device, which is most frequently brought into contact with the patient's mouth and where one is therefore more likely to find residues of blood, saliva and other bacterial colonies from which they come.

In addition, repeated use of saturated or superheated steam at high pressure can cause accelerated aging of the mechanical components of the handpiece.

Thus there exists a need for cleaning and disinfection solutions free of these known limitations.

### SUMMARY OF INVENTION

The invention has as an object to provide a system and a method of cleaning and disinfection which are more efficient than those of the prior art.

Another object of the present invention is to provide a system and a method of cleaning and of disinfection which are less aggressive for the mechanical components of the pieces to be treated compared with solutions of the prior art.

Still another object of the present invention is to provide a solution minimizing the use of chemical substances commonly used for cleaning and disinfection.

Lastly, an object of the present invention is to provide a new solution making it possible to reduce the duration of cleaning and disinfection cycles of the interior of the devices.

These objects are achieved, according to the invention, by means of a system for cleaning and/or disinfecting a tool for dental or surgical use, comprising a steam generator and a compressed air source, the cleaning and/or disinfecting system further comprising a flow regulating device designed to inject wet steam and compressed air into at least one channel of the tool for dental or surgical use that is to be treated.

An advantage of the new system proposed according to the present invention is that it makes it possible to ensure an efficient automatic cleaning of the inside of the dental devices avoiding any accumulation of biofilms, blood and protein residues inside the tool to be treated.

Another advantage of the new system proposed according to the present invention is that it is able to ensure a substantially homogeneous disinfection of the entire dental device to be treated, including the head, i.e. the front part of the tool.

An additional advantage of the new system proposed according to the present invention is to limit the ageing of mechanical components, such as ball bearings or joints (O-rings) and to minimize the corrosion of metal parts compared with conventional automatic cleaning/disinfection procedures using thermos-disinfectors or saturated or superheated steam disinfectors.

Still another advantage of the new system proposed according to the present invention is to eliminate or in any case minimize the use of chemical substances for the cleaning and disinfecting operation.

Still another advantage of the new system proposed according to the present invention is to reduce the duration of cleaning/disinfection cycles of the interior of the devices.

According to a preferred embodiment, the system for cleaning and/or disinfecting a tool for dental or surgical use further comprises a fixing support for the tool for dental or surgical use to be cleaned and/or disinfected, the fixing support being provided with a coupling nose as a substitute for a motor part, and the wet steam-air mixture is able to be injected directly via said coupling nose.

The advantage of such a solution is that it can be easily adapted to existing cleaning and disinfecting devices by very simply adding a module to generate the desired wet steam and steam flows to be connected to the back of handpiece handles.

According to a variant of this preferred embodiment, <in> the system for cleaning and/or disinfecting a tool for dental or surgical use, the fixing support is disposed in a closed enclosure, which makes it possible to easily place a tray for collecting water and waste extracted from the dental or surgical device and to easily recover them without risk of dispersion, on the one hand, and, on the other hand, to add an air filtering system to avoid the ejection of microbiological contaminants outside the enclosure. In this way, the cleaning and disinfection procedure can be optimized from a health point of view.

According to another variant of this preferred embodiment, the enclosure of the cleaning and/or disinfecting system for a tool for dental or surgical use is provided with a system of disinfection with UVC rays.

The advantage of such a variant is to ensure the disinfection of the external surfaces of the dental or surgical device as well as the disinfection of the enclosure itself, thus making it possible to avoid the risk of mutual contamination between devices cleaned and disinfected simultaneously or successively in the same enclosure.

According to another preferred embodiment, the system for cleaning and/or disinfecting a tool for dental or surgical use further comprises a user interface making it possible to determine at least one input parameter selected from among temperature, pressure, wet steam fraction and/or compressed air fraction.

The advantage of such a solution is to be able to optimize to the utmost the desired mixture according to the mechanical or electrical constraints of the system to be cleaned/disinfected and/or the operating procedure performed (dentistry or surgery) prior to cleaning/disinfection, and thus to the regulatory need to carry out, following cleaning/disinfection, a subsequent sterilization in autoclave.

According to another preferred embodiment, the flow regulating device of the proposed system for cleaning and/or disinfecting for a tool for dental or surgical use comprises a first valve for the outlet of wet steam and a second valve for the outlet of compressed air, the second valve being self-regulating.

The advantage of such a solution is to make it possible to minimize the number of system components, not requiring any dedicated chamber to perform the mixing, each of the air and steam flows being fed separately to the tool. Another advantage is also to maximize the efficiency of the cleaning and disinfection cycles, since it is not necessary to achieve an air-steam mixture in thermodynamic equilibrium or quasi-equilibrium (that is to say, a stationary state in time, but maintained out of equilibrium by the flows arriving continuously in the mixing chamber) before the injection in the tool to be treated.

According to a variant of this preferred embodiment, the flow regulating device of the proposed system for cleaning and/or disinfecting for a tool for dental or surgical use further comprises a bypass channel connected to said second valve, the bypass channel being intended to convey an auxiliary compressed air flow to a pneumatic drive system driving in rotation at least some of the rotating parts of the tool for dental or surgical use to be treated.

The advantage of such a solution is, first of all, to optimize the efficiency of the cleaning and disinfection operations of the usually moving parts of the tool, and then to carry out this operation of driving the parts in rotation without requiring a dedicated motor for this purpose, and thus optimizing at the same time the complexity and the needs of the device in terms of power supply.

According to another preferred embodiment, the flow regulating device of the proposed system for cleaning and/or disinfecting for a tool for dental or surgical use further comprises a mixing chamber for making a wet steam-air mixture, which is able to be delivered via a third valve.

The advantage of such a solution is to impose homogeneous conditions inside the device to be cleaned and disinfected, in equilibrium or quasi-equilibrium (i.e. a stationary state in time, but maintained out of equilibrium by the flows arriving continuously in the mixing chamber) thermodynamically, and thus to optimize the conditions of temperature, humidity and pressure according to the needs (flow speed, prioritization of the effectiveness of the cleaning over that of the disinfection etc.).

The present invention relates furthermore to a method for cleaning and/or disinfecting a tool for dental or surgical use using a system for cleaning and/or disinfecting according to the invention, characterized in that it comprises a first step of generating a first flow of wet steam and a second step of generating a second flow of compressed air.

The advantage of such a cleaning and/or disinfection method is that it is possible to combine the different steps in a flexible way between two variants where they are ordered in a completely sequential way or carried out simultaneously.

According to a preferred embodiment, the method for cleaning and/or disinfecting a tool for dental or surgical use according to the invention uses however at least partially simultaneous first and second steps. Thus the durations of the treatment cycle are reduced.

According to a preferred embodiment for the method for cleaning and/or disinfection, the wet steam comprises at least 10% water in liquid phase in the form of droplets.

An advantage of this solution is that it defines a good compromise between the desired cleaning and disinfecting properties, as the steam particles can reach the most hollow and inaccessible areas and condense to dissolve residues and detach biofilm, while the concentration of water in the steam phase and the size of the water particles (‘droplets’) at a lower temperature make it possible to dissolve blood and proteins and transfer biofilm residues to the outside.

According to a preferred embodiment for the method for cleaning and/or disinfecting according to the invention, the effective pressure of the wet air-steam mixture is less than 4.5 bar, so as not to damage the tools to be treated.

According to a preferred embodiment for the method for cleaning and/or disinfecting according to the invention, the partial pressure of the compressed air fraction is between 1.5 and 3 bar. Thus, it is possible to sustain a homogeneous flow of steam without the need to excessively increase the pressure of the steam, thanks to the contribution of the air.

According to a preferred embodiment for the method for cleaning and/or disinfecting according to the invention, the partial pressure of the wet steam fraction is between 2 and 4 bar.

The advantage of such a solution is to be able to maintain a constant flow through the device without causing excessive heating of the surfaces since the steam generation temperature increases with the applied pressure.

According to a preferred embodiment for the method for cleaning and/or disinfecting according to the invention, the wet steam fraction of the wet air-steam mixture is always higher than 50%, so as not to have to generate excessive steam pressures and temperatures if one wants to remain within a range of temperature values sufficient to carry out the cleaning and disinfection operation—always on the order of 100° C.—the effect of the air generally being to cool the mixture.

### BRIEF DESCRIPTION OF THE DRAWINGS

Other advantages and features emerge more clearly from the description which follows of embodiments for implementing the invention, given as non-limiting examples and illustrated in the attached drawings, including:
FIG. 1 is a diagram illustrating the operation of a cleaning and disinfecting device according to a preferred embodiment of the invention;
FIG. 2 is a diagram illustrating the operation of a cleaning and disinfecting device according to a variant of the preferred embodiment of FIG. 1;
FIG. 3 is a diagram illustrating the operation of a cleaning and disinfecting device according to another preferred embodiment of the invention;
FIG. 4 is a diagram illustrating a plurality of possible sequences for the cleaning and disinfection cycles according to the invention;
FIG. 5 is a diagram illustrating how the combination of water and wet steam can advantageously lower the temperature of a mixture used for cleaning and disinfecting the inside of a tool in the context of the present invention.

### DESCRIPTION OF PREFERRED EMBODIMENTS OF THE INVENTION

According to the state of the art, the quality of the steam or vapor in terms of disinfection has always been considered to be inversely proportional to its degree of humidity, and on the contrary directly proportional to the pressure applied. Indeed, humidity is likely to limit the amount of heat transmitted to the bacteria, while one of the advantages of the pressure applied to the steam was to be able to combine a thermal action with a mechanical cleaning action, linked to the mechanical energy of a fluid at high pressure.

Within the framework of the present invention, the technical advances in the characterization of wet steam and of the mixtures between steam and compressed air have been used to gain a better knowledge of the size of the water droplets in the steam and to better control the thermodynamic balances of the steam-water-air mixtures, thus making use of the characteristics of the steam-water-air mixtures, which have always been considered as deficiencies up to now, in order to simultaneously ensure:
1. a mechanical cleaning action
2. chemical dissolution of organic residues
3. gentle detachment of biofilms from metal surfaces
4. homogeneous cleaning and disinfection properties throughout the device from the back to the head, thus avoiding significant temperature and humidity gradients.

Admittedly, the technical feasibility of mixing steam with air was already known in autoclave technology in the context of sterilization processes of devices already containing saline solutions or other substances; the aim in this case, however, was to be able to sterilize the outer casing of pressurized components, requiring a constant pressure environment necessitating the creation of a partial vacuum prior to the introduction of the steam. For these systems, however, the air-steam mixing is performed under quasi-stationary conditions and close to thermodynamic equilibrium (similar pressure and temperature), precisely to avoid excessive pressure gradients and any flow.

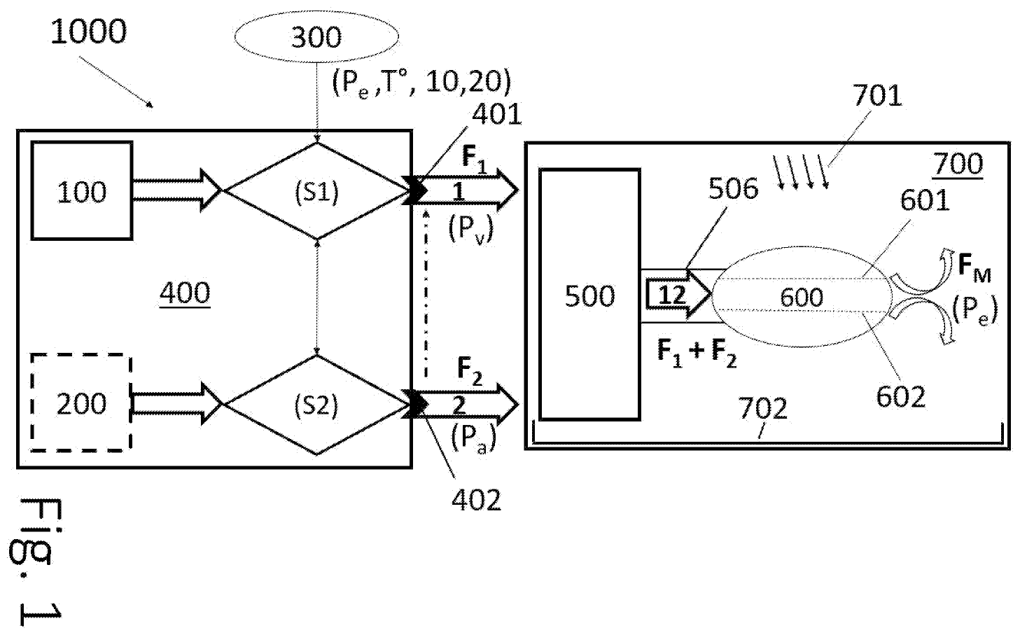

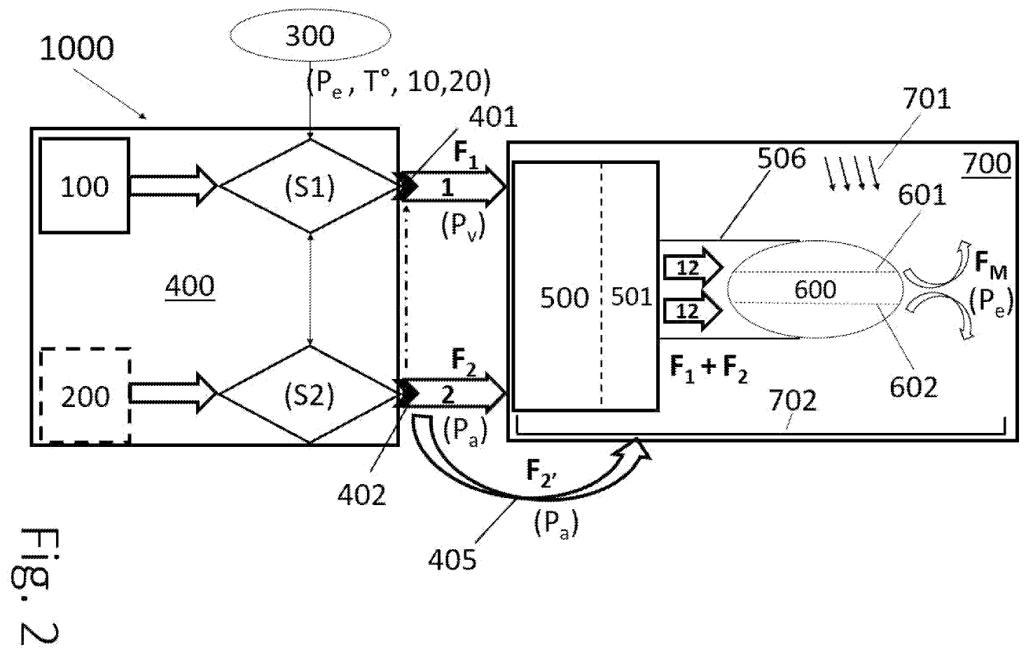

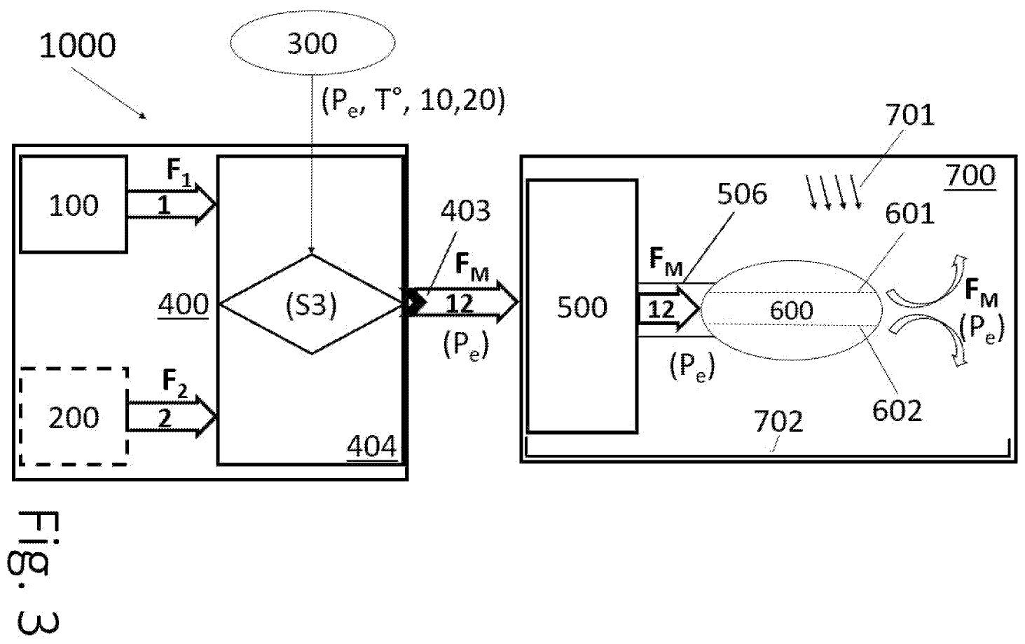

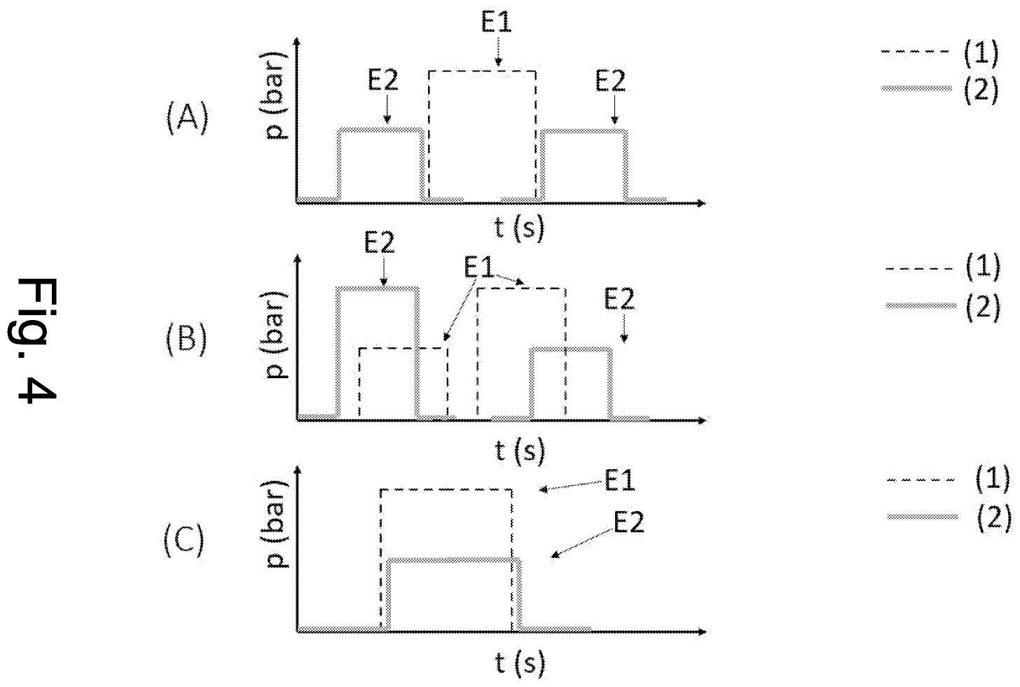

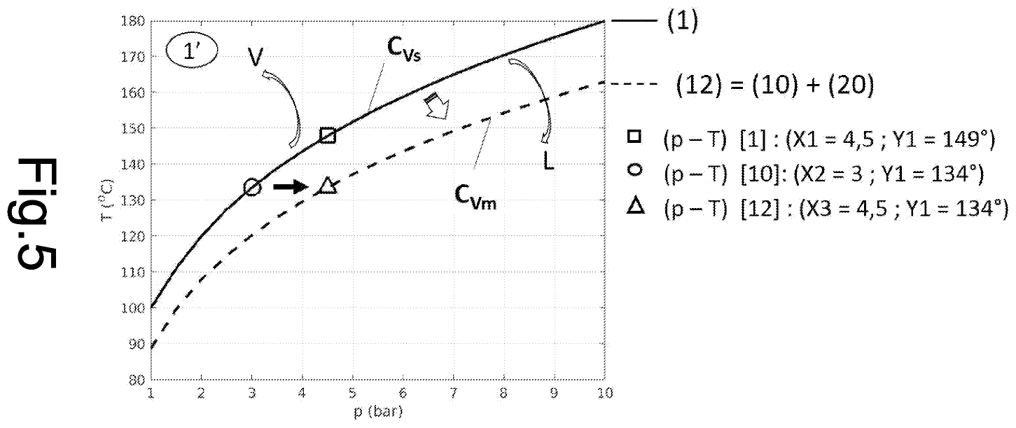

In the following, it will be shown how it is possible to overcome in particular the technical prejudice according to which the use of wet steam would be unsuitable for both a cleaning and a disinfecting process of the interior of a piece to be treated, and how it is possible to carry out a mixture to optimize separately the pressure and the temperature of the steam, even if it is saturated or close to the level of saturation indicated by the curves of saturating steam pressure of FIG. 5 discussed hereinafter, for which there is a strict relationship between the pressure P and the temperature T.

FIG. 1 shows an operating diagram of a cleaning and disinfecting system 1000 according to a preferred embodiment for the present invention, which aims to inject wet steam 1 into the interior of dental devices to be treated—tools 600—from their coupling part, i.e. their rear part, corresponding in principle to the handle of a handpiece. According to this preferred embodiment, the coupling part is formed by a standard coupling nose 506 arranged on a fixing support 500 arranged in a closed enclosure 700, i.e. capable of being isolated from the outside. The fixing support 500 of the enclosure 700 is provided here for arranging a single tool to be cleaned and disinfected (the speed of the operation performed by the system according to the invention allows this); however, it could alternatively be possible to arrange a plurality of tools to be cleaned and disinfected at the same time for reasons of economy of scale and if the type of clinical treatment allows it (for example if all the tools have been used to treat the same patient). The enclosure 700 also preferably includes a disinfection system 701 using UVC rays (subset of ultraviolet rays, spectral band “C”), in order to disinfect the exterior of each of the tools to be treated and thus avoid any mutual self-contamination. In order to facilitate the cleaning process of the enclosure 700 itself, a removable collection tray 702 is provided to collect the water and waste extracted from the tools 600. In addition, an air filtering system (not shown) could also be provided to avoid any ejection of microbiological contaminants outside the enclosure.

In the present invention, it is sought to insert compressed air 1 simultaneously or alternatively to the insertion of wet steam 2 inside the dental devices from the coupling part, and the air-wet steam mixture 12 then emerges from the tool head 600, on the right of the figure.

In the operating diagram shown in FIG. 1, it can be seen that the first flow F1 of wet steam 1 and the second flow F2 of compressed air are kept separate until they arrive at the delivery line to the device, located here inside the coupling nose 506 provided on the fixing support 500. There is thus no mixing chamber for the two phases, which makes the proposed cleaning and disinfection system 1000 particularly simple to implement, and not very bulky. The first flow F1 of wet steam 1 is added to the second flow F2 of air to generate a “combined” flow F1+F2 inside the channels of the tool 600, consisting for example of a central cooling channel 601 and one or more spray channels 602 intended to convey air and/or water to the working area near the head of the tool 600. There is thus no thermodynamic equilibrium at the inlet of the tool 600 to be treated between the first flow F1 of wet steam 1 sent at a partial pressure whose value Pv is potentially different from that of Pa and the second flow F2 of compressed air, which is moreover injected at a temperature generally close to the ambient temperature (i.e. around 20 degrees), i.e. much lower than that of the steam, rather close to 100 degrees Celsius. However, this equilibrium can be approached at the head of the tool 600, and this is the reason why at the level of the arrows which emerge from it, the flow corresponding to the wet steam-air mixture 12 is indicated by the reference FM, and to which a pressure value Pe is supposed to be applied. This pressure value Pe corresponds to the pressure that can be defined at a user interface 300 of an upstream flow regulating device 400, and which defines the effective pressure of the mixture, i.e. taking into account not only the partial pressures of each of the air-vapor phases, but also their respective fraction and volume in the mixture.

The user interface 300 preferably comprises a control member such as a button or an input interface such as a touch screen, and also a display module for checking the input before validating the setting. According to the preferred embodiment described, an effective pressure value Pe will preferably be chosen which can, depending on the fractions of steam and compressed air in the mixture, define a temperature suitable for the cleaning and disinfection operation. Nevertheless, the input parameters are not limited to an effective pressure value Pe, but can also include a temperature value T°, and also adjust the percentages related to the compressed air fraction 20 and the wet steam fraction 10.

The flow regulating device 400 comprises a steam generator 100 as well as an integrated or external compressed air source 200, as well as a first valve 401 from which the first flow F1 of wet steam 1 is generated at the outlet and a second valve 402 from which the second flow F2 of compressed air is generated at the outlet. The opening and closing of each of these valves is carried out respectively following control steps using, for example, associated sensors or simply via programmable timers, predefined as a function of the control parameter (effective pressure Pe or temperature T° or moisture fraction of the steam). In FIG. 1, the steam pressure control step is referred to as step S1, while the compressed air pressure control step is referred to as S2. However, it can be seen from this figure that according to this preferred embodiment, the second control step S2 is actually dependent on the first control step S1, because by selecting an effective pressure value Pe for the mixture 12 containing wet steam and air, and for predetermined proportions of air and wet steam, the partial pressure of air Pa will then be automatically defined by that of steam Pv. In other words, the second valve 402 will in this case be self-regulated according to the first valve 401, hence the arrow indicating the connection between the two control steps S1, S2 as well as the dotted arrow indicating the alignment of the second valve 402 on the first valve 401.

FIG. 2 illustrates another preferred embodiment for the cleaning and/or disinfecting system 1000 for the present invention, which consists of a variant of the first embodiment of FIG. 1. For the sake of simplicity, not all common elements will be described again in detail; in particular the left-hand side of the figure relating to the flow regulating device 400 which is completely identical.

The improvement of the cleaning and/or disinfecting system 1000 proposed according to this variant consists in providing a bypass channel 405 at the outlet of the second valve 402, so that the compressed air 2 can be delivered simultaneously to two separate pipes, one being identical to the previous one for conveying the second flow of compressed air F2 to the tool, and henceforth another conveying an auxiliary flow of compressed air F2′ intended to fulfil a driving function for certain rotating parts of the tool. Thus, while a first pipe brings the second compressed air flow F2 together with the first wet steam flow F1 inside the coupling nose 506 and penetrating into the spray pipes 602 and the cooling channel 601 of the tool, the generated auxiliary compressed air flow F2′ is sent via the bypass channel 405 at an air partial pressure of value Pa, a priori identical to that injected into the coupling nose 506 of the tool attachment device, in order to actuate a pneumatic drive device 501 mechanically connected to the fixing support 500, such as a propeller turbine. The action of the compressed air will allow the coupling nose support 506 to rotate slowly, thus, via the mechanical link, the gear chain of the straight handpiece or the contra-angle. And when the support 500 comprises a plurality of coupling noses 506 intended to receive a plurality of tools 600 to be treated, it will be possible in this case to provide dedicated driving devices 501 for each coupling nose 506, i.e. to actuate in rotation effectively each of the gear chains of each tool 600. To do this, several kinematic chains can be mechanically connected in parallel to the same propeller, or alternatively, specific air flows can be dedicated to propellers each connected to a corresponding coupling nose 506 via bypasses.

According to the previously described methods, steam and air are injected directly into the pipe coming out into the tool 500 to be cleaned/disinfected, thus creating a mixture that is not in thermodynamic equilibrium. This configuration requires fewer components, but is not optimal in terms of the homogeneity of the mixture obtained.

In the following, a further preferred embodiment involving a mixing chamber separate from the pipe opening into the tool 500 to be cleaned/disinfected will be described. In this case, the mixture of wet steam—air 12 can be achieved close to thermodynamic equilibrium or at least a stationary condition of quasi-equilibrium (by balancing pressure and temperature of the components around a ratio close to 1, for example between 0.7 and 1.3), which allows possibly part of the condensed water to be collected before injecting in the tool not only a mixture containing air and wet steam (combination of saturated steam and water droplets in suspension or ‘droplets’), but also at least partially water in the form of droplets that are not in suspension . In this case, the presence of a system to regulate the pressure of the mixture to be injected into the device is necessary, but it also makes it possible to regulate more precisely the flow of the mixture intended for the cleaning/disinfection.

FIG. 3 illustrates an operating diagram for such an embodiment; as for the previous FIG. 2, only the elements that differ from FIG. 1 will be described in detail, and reference will be made to the latter for all the elements common to it.

As can be seen on the left side of FIG. 3, the steam generator 100 and the compressed air source 200 integrated or external to the flow control device 400 deliver a first flow F1 of wet steam 1 and a second flow F2 of compressed air to a mixing chamber 404 of the two phases. Here, it is the effective pressure Pe of the wet steam-air mixture 12 that can be selected via the user interface 300, and a third flow FM corresponding to the wet steam-air mixture 12 is generated at the outlet of a third valve 403 following a third step of control S3 of the pressure of this wet steam-air mixture and/or of programmable time delay and defined by the adjustable parameter. This third flow FM of wet steam-air mixture 12 is introduced in thermodynamic equilibrium into the pipes of the tool to be treated (cooling channel 601 and spray channels 602) at the level of the coupling nose 506. This embodiment is more easily controllable since it no longer involves self-adjustment of the air pressure) and makes it possible to obtain a more homogeneous mixture inside the fluid supply line to the device to be treated, i.e., the tool 600, and thus in the tool itself; it is, however, obviously less compact in terms of space requirements than the first embodiment described in connection with FIG. 1, due to the volume of the mixing chamber.

Although not illustrated, a variant similar to that shown in FIG. 2 may be provided for this mode of implementation by providing a direct supply of compressed air to the support of the tool or tools to be treated in order to produce a rotational movement under the pneumatic action of a turbine. Nevertheless, the bypass channel will have to be arranged upstream of the mixing chamber 404.

FIG. 4 shows three non-limiting examples of the pressure profile of the wet steam 1 and the compressed air 2:
in example (A) corresponding to the top figure, air 2 and wet steam 1 are introduced sequentially, i.e., one after the other without temporal overlap: first compressed air 2 to cool the dental/surgical device, then wet steam 2 <sic. 1> to clean, then compressed air 2 again to dry and cool the tool again.
In the example (B) corresponding to the middle figure, the air 2 and the wet steam 1 are introduced not simultaneously, but with at least partial temporal overlap. The pressure of the air 2 and the wet steam 1, as well as the ratio between them, are not constant during the whole process; in the case described this ratio changes from a value lower than 1 to a value higher than 1.
In the example (C) corresponding to the lower figure, air 2 and wet steam 1 are introduced substantially simultaneously (90% or more temporal overlap).

Represented in FIG. 4 are a first step E1, which corresponds to the generation of a first flow F1 of wet steam 1 to be injected directly or indirectly into the tool to be cleaned and/or disinfected, such as those shown in FIGS. 1 to 3, and similarly a second step E2, for the generation of a second flow F2 of compressed air 2. These two steps can be adjusted temporally with respect to each other as required.

In cases where wet steam 1 and air 2 are generated via simultaneous flows, a mixture 12 containing wet steam and air is realized, the thermodynamic properties of which can be summarized as follows:
1. P=Pa+Pv: the total pressure is the sum of the two pressures
2. Pe=P Vv/(Vv+Va): the effective pressure of the wet steam—air mixture 12 is reduced in relation to the total pressure by a factor which depends on the volumes of each of the two fluids (respectively air and steam, their volumes being denoted Va and Vv).

These two relationships above show that it is possible to obtain steam at high pressure with reduced temperature. For example, a mixture composed of two thirds of steam at 3 bar (absolute pressure) and one third of air at 1.5 bar (absolute) produces a mixture at 4.5 bar at about 134° C., which is ideal for disinfection without inducing excessive heating of the device to be disinfected, whereas pure steam at 4.5 bar would be close to 150° C., and therefore potentially harmful to certain mechanical components.

This example is shown in FIG. 5, illustrating two saturating vapor pressure curves respectively for saturated steam 1, and respectively a mixture 12 containing saturated steam and compressed air. For each of these curves, there is a strict relationship between the pressure P and the temperature T° corresponding to a line of demarcation corresponding to the phase change between the liquid phase “L” and the vapor phase “V”:
The CVs curve relating to pure saturated steam starts at 100° C. under atmospheric pressure of 1 bar, to reach a little less than 150° C. under a pressure of 4.5 bar [point of coordinates (X1,Y1) on this curve indicated by a square].
The CVm curve relative to a mixture comprising a fraction of wet steam 10 equal to 66.6% and a fraction of compressed air 20 also equal to 33.3% to form a steam-wet-air mixture 12 starts at around 90° C. under atmospheric pressure, and reaches only 134° C. under a pressure of 4.5 bar [point of coordinates (X3,Y3) on this curve indicated by a triangle], this temperature corresponding to a saturated steam under a pressure of 3 bar [point of coordinates (X2,Y2) on the preceding curve CVs indicated by a circle].

It can thus be easily seen that the pressure-temperature curve of a mixture with 33% compressed air 2 is about 15° C. below the curve of pure saturated steam CVs. The mixture of wet steam at 3 bar and air at 1.5 bar thus makes it possible to increase the pressure to 4.5 bar (and thus to maximize the mechanical effect of the cleaning), without needing to increase the temperature excessively: an equivalent pressure would not be achievable with pure steam without greatly exceeding the usual sterilization/disinfection temperature (149° C. vs 134° C.). The arrow pointing from the CVs curve to the substantially downward sloping CVm curve illustrates how the shift in the p-T° relationship between these two curves makes it possible to achieve this beneficial temperature reduction.

It will be understood from the above that using this technique of “diluting” wet steam with air, it would be possible to obtain wet steam-air mixtures 12 for cleaning approaching 100° C. by correspondingly increasing the pressure and further increasing the contribution of air in the mixture, i.e. increasing the percentage of the compressed air fraction 20. However, in order to remain in working conditions where the temperature remains sufficiently high to maintain effective disinfecting properties, but without generating too high a pressure that could damage the tools to be treated, a fraction of wet steam 20 should always be greater than 50%. For the same reasons, the effective pressure Pe of the mixture should be less than 4.5 bar, and the partial pressure corresponding to the wet steam fraction 10 should always be less than 4 bar. According to a particularly preferred embodiment, the rate of the wet steam fraction 20 is between 50% and 66% of the total mixture.

Furthermore, in order to maximize the cleaning properties of the wet air-steam mixture 12 obtained, it may be possible to obtain wet steam 1 comprising at least 10% water in the liquid phase in the form of droplets. In other words, we will be slightly below the curve defining the saturation vapor pressure of the mixture 12, i.e. on the side where the liquid phase is supposed to become predominant again. The arrow pointing downwards and the “L” indicates the direction of the phase change towards the liquid phase, while the arrow pointing upwards and the “V” indicates the opposite direction of phase change towards the vapor phase, the dry steam 1′ corresponding in fact to the whole part of the diagram situated above the saturating vapor pressure curve.

Within the scope of the present invention, following the cleaning/disinfection phase, it would still be possible to add a pure disinfection step, carried out with the injection of superheated dry steam, or of a mixture of superheated dry steam and compressed air, the properties of this mixture being defined according to the procedure described in FIG. 5. In fact, following an efficient cleaning, the disinfection efficiency of the superheated dry steam would be strongly improved, which allows the use of moderate pressure values, typically below 4.5 bar and advantageously below 3.5 bar, to better preserve the dental or surgical tools to be treated.

Advantageously, the cleaning and disinfection device can be equipped with an integrated water tank, to be filled with distilled, demineralized or tap water if the hardness allows it. In this case, the optimum volume of the water tank is between 0.3 I and 0.7 I, to optimize the number of cleanings/disinfections that can be performed before filling the tank (one filling per week, at a rate of 30 cleanings/disinfections per day, being optimal) and to minimize the dimensions of the device.

The detailed description of the operation of the proposed new system according to a preferred embodiment has been made in the case of a coupling nose for contra-angle or straight handpieces. In the case where the tool to be cleaned and disinfected is a turbine, the only differences are related to the geometry of the coupling nose, which will take the form of a turbine coupling, and in the means of setting the rotor in motion, which in this case is set in motion simply by the passage of the pressurized fluid (wet steam, compressed air or a mixture of the two fluids)

It will therefore be understood from the foregoing that the surgical dental tool 600 to be treated may be either a contra-angle, a straight handpiece, or a turbine.

Furthermore, in the context of the present invention, reference is made to a steam generator 100 for generating a first flow of wet steam F1 to be injected, directly or indirectly, i.e., via prior mixing or not with a second air flow F2, into the tool 600 to be treated. It will be understood that such a steam generator 100 is not limited to a saturated wet steam generator, but may also include an at least partially dry steam generator, provided that the resulting steam flow injected into the tool, or exiting from the head of the treated tool, contains wet steam, the underlying idea of the invention being precisely to use wet steam for the cleaning and/or disinfecting operation, and no longer only dry steam.

1. System for cleaning and/or disinfecting a tool for dental or surgical use, comprising a steam generator, and a compressed air source, and, a flow regulating device configured to inject wet steam and compressed air into at least one channel of the tool for dental or surgical use that is to be cleaned and/or disinfected.

2. System for cleaning and/or disinfecting a tool for dental or surgical use according to claim 1, further comprising a fixing support for said tool for dental or surgical use to be cleaned and/or disinfected, said fixing support being provided with a coupling nose as a substitute for a motor, and said wet steam-air mixture being injectable directly via said coupling nose.

3. System for cleaning and/or disinfecting a tool for dental or surgical use according to claim 2, wherein said fixing support is disposed in a closed enclosure.

4. System or cleaning and/or disinfecting a tool for dental or surgical use according to claim 3, said enclosure being provided with a disinfection system with UVC rays.

5. System for cleaning and/or disinfecting a tool for dental or surgical use according to claim 1, further comprising a user interface making it possible to determine at least one input parameter selected from among temperature, pressure, wet steam fraction and/or compressed air fraction.

6. System for cleaning and/or disinfecting a tool for dental or surgical use according to one of the preceding claim 1, wherein the flow regulating device comprises a first valve for outlet of wet steam and a second valve for outlet of compressed air, said second valve being self-regulating.

7. System for cleaning and/or disinfecting a tool for dental or surgical use according to claim 6, wherein the flow regulating device further comprises a bypass channel connected to said second valve, said bypass channel being configured to convey an auxiliary compressed air flow to a pneumatic drive system driving in rotation at least some rotating parts of said tool for dental or surgical use.

8. System for cleaning and/or disinfecting a tool for dental or surgical use according to claim 1, wherein the flow regulating device comprises a mixing chamber configured to make a wet steam-air mixture, which is deliverable via a third valve.

9. System for cleaning and/or disinfecting a tool for dental or surgical use according to claim 1, wherein the flow regulating device is configured to inject at least partially simultaneously wet steam and compressed air into at least one channel of said tool for dental or surgical use that is to be cleaned and/or disinfected.

10. Method for cleaning and/or disinfecting a tool for dental or surgical use using the system for cleaning and/or disinfecting according to claim 1, the method comprising a first step of generating a first flow of wet steam and a second step of generating a second flow of compressed air.

11. Method for cleaning and/or disinfecting a tool (600) for dental or surgical use according to claim 10, said first step and second step being at least partially simultaneous.

12. Method for cleaning and/or disinfecting a tool for dental or surgical use according to claim 10, said wet steam comprising at least 10% water in liquid phase in the form of droplets.

13. Method for cleaning and/or disinfecting a tool for dental or surgical use according to claim 10, an effective pressure of said wet air-steam mixture being less than 4.5 bar.

14. Method for cleaning and/or disinfecting a tool for dental or surgical use according to claim 10, wherein a partial pressure of the compressed air is between 1.5 and 3 bar.

15. Method for cleaning and/or disinfecting a tool for dental or surgical use according to claim 10, wherein a partial pressure of the wet steam is between 2 and 4 bar.

16. Method for cleaning and/or disinfecting a tool for dental or surgical use according to claim 10, wherein a wet steam fraction of said wet air-steam mixture is greater than 50%.
